(19) United States
(12) Patent Application Publication
(10) Pub. No.: US 2026/0157814 A1
(43) Pub. Date: Jun. 11, 2026

(54) GRAPHIC USER INTERFACE FOOT PEDALS FOR A SURGICAL ROBOTIC SYSTEM

(71) Applicant: Covidien LP, Mansfield, MA (US)

(72) Inventor: Arvind K. Ramadorai, Lexington, MA (US)

(21) Appl. No.: 18/707,645

(22) PCT Filed: Dec. 5, 2022
(86) PCT No.: PCT/US2022/051789

§ 371 Date: May 6, 2024

Related U.S. Application Data
(60) Provisional application No. 63/286,172, filed on Dec. 6, 2021.

(51) Int. Cl. A61B 34/37; A61B 34/00; A61B 34/30

(52) U.S. Cl. CPC A61B 34/37; A61B 34/25; A61B 2034/301

(57) ABSTRACT

Devices, systems, and methods control a movement or instrument function of a robotic arm of a surgical robotic system. The devices include a graphic user interface. The graphic user interface includes one or more foot pedal images on a touchscreen display and assigns a control input for a specific movement or instrument function of the surgical robotic system to the foot pedal images. The graphic user interface is further configured to receive a touch input at a location where the foot pedal images are displayed on the touchscreen display, generate input data based on receiving the touch input, and send the input data to a surgical console of the surgical robotic system.

### CROSS-REFERENCE TO RELATED APPLICATIONS

The application claims priority to U.S. Provisional Patent Application Ser. No. 63/286,172, filed on Dec. 6, 2021, the entirety of which is incorporated herein by reference.

### BACKGROUND

Surgical robotic systems may include a surgical console controlling one or more surgical robotic arms, each having a surgical instrument having an end effector (e.g., forceps or grasping instrument). In operation, a user provides input to the surgical robotic systems through one or more interface devices, which are interpreted by a control tower of a surgical console as movement commands for moving the surgical robotic arm. Based on the user inputs, the surgical console sends movement commands to the robotic arm so that the robotic arm is moved to a position over a patient and the surgical instrument is guided into a small incision via a surgical access port or a natural orifice of a patient to position the end effector at a work site within the patient's body.

### SUMMARY

One embodiment of the present disclosure is directed to a foot pedal system for a surgical robotic system. The foot pedal system includes a graphic user interface. The graphic user interface is configured to display a foot pedal image on a touchscreen display and assign a control input for a specific movement or instrument function of the surgical robotic system to the foot pedal image. The graphic user interface is further configured to receive a touch input at a location where the foot pedal image is displayed on the touchscreen display, generate input data based on receiving the touch input, and send the input data to a surgical console of the surgical robotic system. In aspects the foot pedal system includes a processor, a memory, and a transmitter.

In aspects, the foot pedal image includes shapes, drawings, and pictures within the touch screen display.

In aspects, the foot pedal images include at least one of a left foot image, a right foot image, a toe and heel image, or a drawing or picture of a piece of equipment to be controlled.

In aspects, the foot pedal image includes text.

In aspects, the graphic user interface provides an indication that a touch input has been registered.

In aspects, the indication that a touch input has been registered is haptic feedback, audio feedback, visual feedback, or any combination thereof.

In aspects, the input data is utilized by the surgical console of the surgical robotic system to remotely control the specific movement or instrument function of a surgical robotic arm.

In aspects, the instrument function includes one of bipolar coagulation, tissue cutting, stapling, monopolar power level, or ultrasonic power level.

Another embodiment of the present disclosure is a system for controlling a movement or instrument function of a robotic arm of a surgical robotic system. The system includes a touchscreen display, a surgical console, and the robotic arm. The touch screen display is configured to output a graphic user interface (GUI) including a foot pedal image. The GUI is configured to assign a control input for a specific movement or instrument function of the surgical robotic system to the foot pedal image. The graphic user interface is further configured to receive a touch input at a location where the foot pedal image is displayed on the touchscreen display, generate input data based on receiving the touch input, and send the input data to a surgical console of the surgical robotic system. The surgical console utilizes the input data to remotely control the specific movement or instrument function of the surgical robotic arm.

Another embodiment of the present disclosure is a method for controlling a movement or instrument function of a robotic arm of a surgical robotic system. The method includes displaying a graphic user interface including a foot pedal image on a touchscreen display and assigning a control input for a specific movement or instrument function of the surgical robotic system to the foot pedal image. The method also includes receiving at the graphic user interface a touch input at a location where the foot pedal image is displayed on the touchscreen display, generating input data based on receiving the touch input, and sending the input data to a surgical console of the surgical robotic system. The method further includes the surgical console utilizing the input data to remotely control the specific movement or instrument function of the surgical robotic arm.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a top perspective view of a graphic user interface foot pedal of the surgical robotic system of FIG. 1 according to an embodiment of the present disclosure;

FIG. 6 is a top perspective view of a graphic user interface foot pedal of the surgical robotic system of FIG. 1 according to an embodiment of the present disclosure; and

FIG. 7 is a flow diagram of an example method for controlling a movement or instrument function of a robotic arm of a surgical robotic system according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views. As used herein the term “proximal” refers to the portion of the surgical robotic system and/or the surgical instrument coupled thereto that is closer to a base of a robot, while the term “distal” refers to the portion that is farther from the base of the robot.

As will be described in detail below, the present disclosure is directed to a surgical robotic system, which includes a surgical console, a control tower, and one or more mobile carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

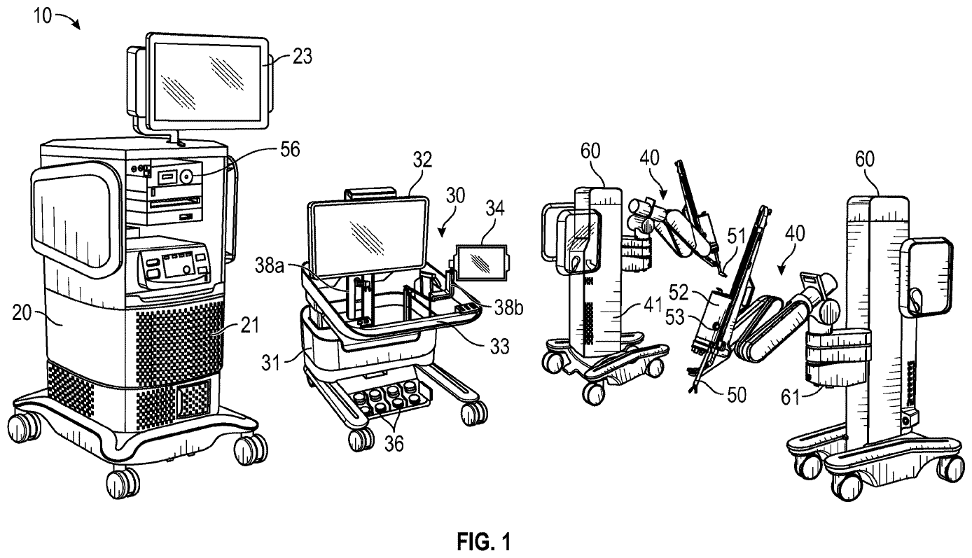

With reference to FIG. 1, a surgical robotic system 10 includes a control tower 20, which is connected to all of the components of the surgical robotic system 10 including a surgical console 30 and one or more movable carts 60. Each of the movable carts 60 includes a robotic arm 40 having a surgical instrument 50 removably coupled thereto. The robotic arm 40 is also coupled to the movable cart 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device configured to receive the video feed from the endoscopic camera 51, perform image processing, and output the processed video stream.

The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as a foot pedal system 36 having a plurality of foot pedals and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedal system 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

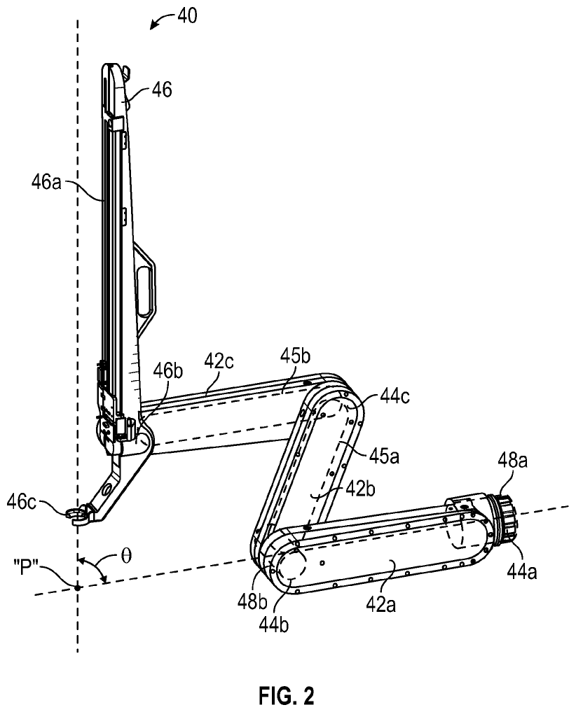

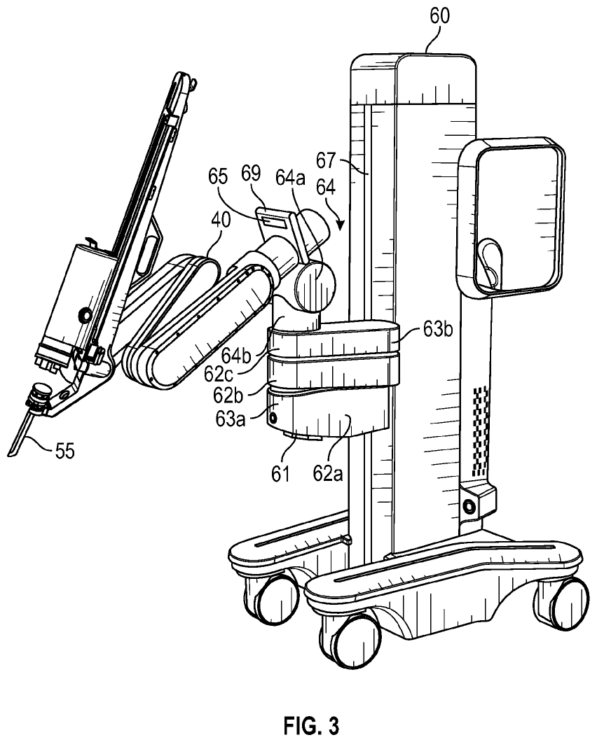

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and is configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46. The holder 46 also includes a port latch 46c for securing the port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

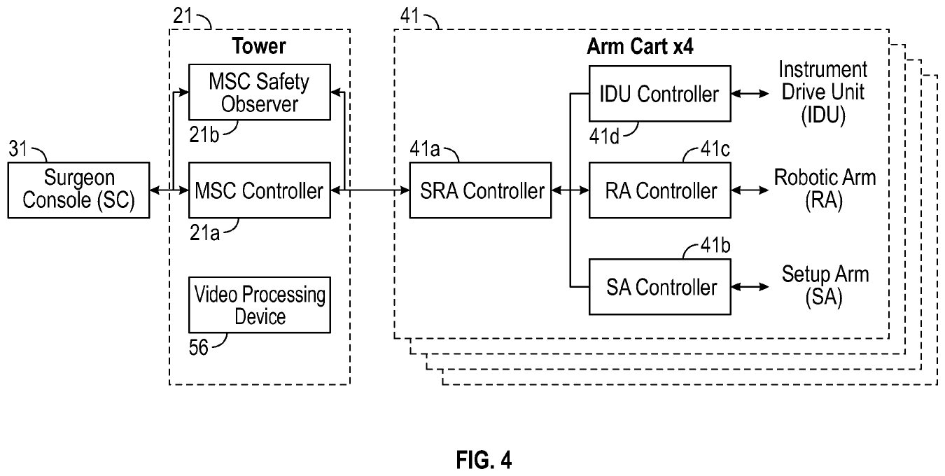

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals of the foot pedal system 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61. Joints 63a and 63b and the rotatable base 64 are stationary when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and roll-pitch-yaw (RPY) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

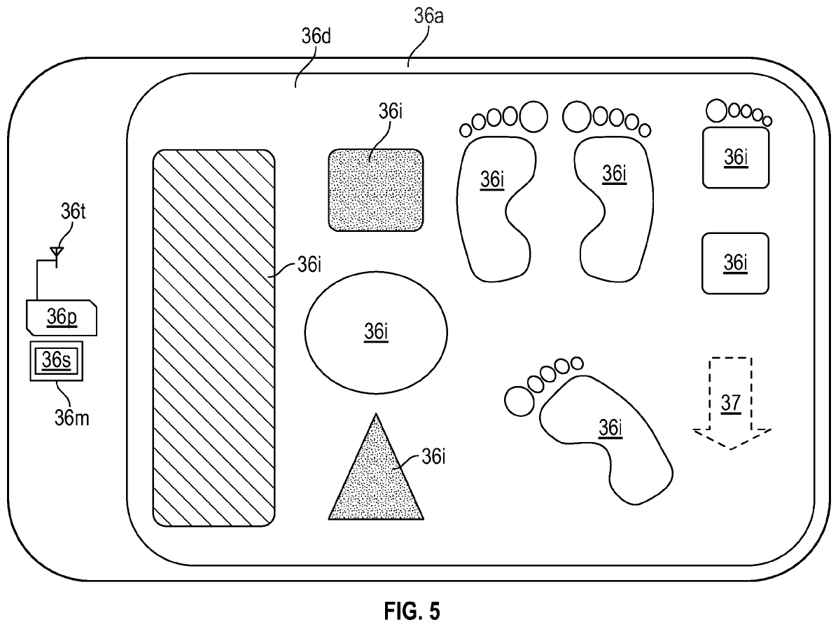

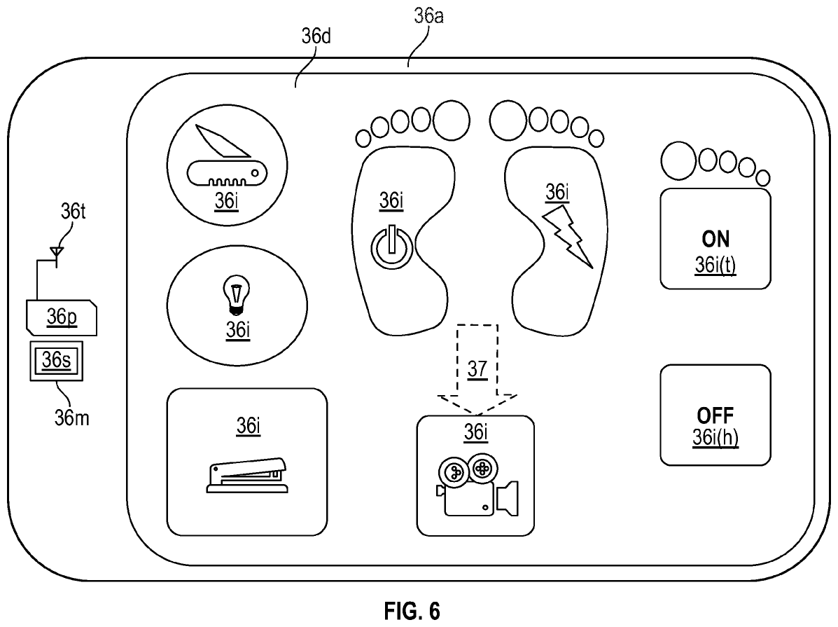

With reference to FIGS. 5 and 6, the foot pedal system 36 also includes a graphic user interface 36a in lieu of or in addition to the foot pedals of FIG. 1. Graphic user interface 36a is displayed on a touchscreen display 36d. The touchscreen display 36d may be any suitable touch-sensing screen including capacitive, resistive, or other touch-sensing panel embedded in the screen, which may be an LCD, AMOLED or OLED. Touchscreen display 36d may be activated by a user's shoe or foot of a user or with a reusable or disposable bootie allowing for registration of inputs and interaction of the user with touchscreen display 36d. The shoes and/or booties may include one or more sensors allowing for the touchscreen display 36d to register location of the user's foot wearing the shoe and/or bootie. In embodiments, user input on the touchscreen display 36d may also be provided with other user appendages.

The touchscreen display 36d is coupled to a processor 36p in communication with a memory 36m, and a transmitter 36t. Memory 36m may include configurable software 36s. Configurable software 36s of graphic user interface 36a, when executed by processor 36p, may allow the user to configure touchscreen display 36d with selectable foot inputs 36i to be displayed at a configurable location within touch screen display 36d. Touchscreen display 36d may be located in a same location or proximate to a user and may be configured to be activated by the user's feet. Unlike conventional foot pedals, a foot pedal system with graphic user interface 36a may allow a user to customize foot pedal controls for a surgical robotic system based on the user's preference, the procedure to be performed, or the user's habitus.

Selectable foot inputs 36i may include various shapes, colors, drawings, pictures, icons, and/or indicia, and be provided in a variety of sizes, locations, orientations, and arrangements within touch screen display 36d. In embodiments, selectable foot inputs 36i may include left and right foot images, toe 36i(t) and heel 36i(h) images, drawings or pictures of a piece of equipment to be controlled, a power symbol, geometric shapes including boxes, circles, ellipses, triangles, and various shape/color/size combinations. Selectable foot inputs 36i may further include text or indicia which may provide context or description to an activity controlled by selectable foot input 36i. Selectable foot inputs 36i may take the form of an image of an actual or physical foot pedal 36 from a physical surgeon console 30, or the like.

Configurable software 36s of graphic user interface 36a may allow the user to assign a control input for a specific movement or instrument function to each selectable foot input 36i displayed on touchscreen display 36d. Configurable software 36s of graphic user interface 36a may allow the user to turn on or off selectable foot inputs 36i displayed on touchscreen display 36d. In embodiments, foot pedal images 36i may include an image for a toe portion of a foot and an image for a heal portion of a foot, and a user may assign a first control input for the toe portion image 36i(t) and a second control input for the heal portion image 36i(h).

In embodiments selectable foot inputs 36i displayed on display 36d may be configured through a graphic user interface of a surgeon's console or a graphic user interface of a control tower.

Touchscreen display 36d may be configured to receive a touch input 37 such as a tap, swipe, slide, etc., from the user when a touch is made at a location where selectable foot input 36i is displayed on touchscreen display 36d. Touchscreen display 36d may be configured to provide an indication that a touch input 37 has been registered by touchscreen display 36d. An indication that a touch input 37 has been registered by touchscreen display 36d may include haptic feedback, audio feedback, visual feedback, and combinations thereof.

Processor 36p is configured to generate input data based on receiving touch input 37 from touchscreen display 36d. Processor 36p of graphic user interface 36a may send input data to surgical console 30, control tower 20 and/or at least one of the robotic arms 40 of FIG. 1 by transmitter 36t or other communication protocols of the system 10 described above. While a wireless transmitter 36t is illustrated, it is contemplated and within the scope of the disclosure that hardwired connections may be used to send input data to the surgical console 30 the control tower 20 and/or the robotic arm(s) 40. Input data from graphic user interface 36a may be utilized by surgical console 30 to remotely control a specific movement or instrument function of robotic arms 40 of FIG. 1. Instrument function remotely controlled by input data from graphic user interface 36a may include bipolar coagulation, tissue cutting, stapling, monopolar power level, ultrasonic power level, etc. A foot pedal system with graphic user interface 36a may include an endoscope mode to allow a user to control an endoscope associated with system 10 or endoscopic camera 51 of FIG. 1 with selectable foot inputs 36i and input data from graphic user interface 36a.

Arrangement of selectable foot inputs 36i displayed on touchscreen display 30d may be based on a preference of the user, a procedure to be performed, the user's habitus (short legs, long legs), or other factors. Placement of touchscreen display 36d of graphic user interface 36a may be based on a preference of the user. In embodiments, the graphic user interface 36a may be positioned higher or lower on the touchscreen display 36d, father away or closer to, and/or left or right of the touchscreen display 36d based on a preference of the user. Touchscreen display 36d may be configured to receive touch input 37 where selectable foot inputs 36i are displayed and may provide data to surgical console 30, control tower 20 and/or robotic arm(s) 40 through transmitter 36t to be utilized to remotely control one or more robotic arms 40 of FIG. 1.

Touchscreen display 36d may be portable and a position of touchscreen display 36d relative to a user may be customizable. In embodiments, touchscreen display 36d of graphic user interface 36a may be positioned higher or lower, father away or closer to, and/or left or right of a user based on a preference of the user. In an embodiment, touchscreen display 36d may include multiple touchscreens 36d as a foot station, and the multiple touchscreens 36d may be at different heights such as an upper touchscreen 36d and a lower touchscreen 36d. Configurable software 36s may allow a user to move selectable foot inputs 36i from one touchscreen display 36d to another touchscreen display 36d within a multiple touchscreen embodiment. Configurable software 36s may also allow a user to move selectable foot inputs 36i around within a touchscreen display 36d. In embodiments, configurable software 36s may include a mode for movement of selectable foot inputs 36i which may allow movement and reconfiguration of a selectable foot input 36i based on user's foot motion on touchscreen display 36d and/or a user's hands at a console display such as a graphic user interface of a surgeon's console or a graphic user interface of a control tower. A user may initiate a mode for movement for touchscreen display 36d and then drag a foot on touchscreen display 36d or an appendage on a console display to move and place a selectable foot input 36i at a desired location within touchscreen display 36d.

A device in accordance with the present disclosure may provide a user with the ability to configure foot pedal controls for a surgical robotic system. A device in accordance with the present disclosure may provide a user with the ability to configure foot pedal controls through one of multiple graphic user interfaces of a surgical robotic system including a graphic user interface of a foot pedal device, a graphic user interface of a surgeon's console, or a graphic user interface of a control tower. A device in accordance with the present disclosure may provide a user with the ability to configure foot pedal controls based on the user's preference, the procedure to be performed, or the user's habitus. A device in accordance with the present disclosure may enable multiple foot pedal inputs to be registered through a singular graphic user interface or through one of various graphic user interfaces of a surgical robotic system.

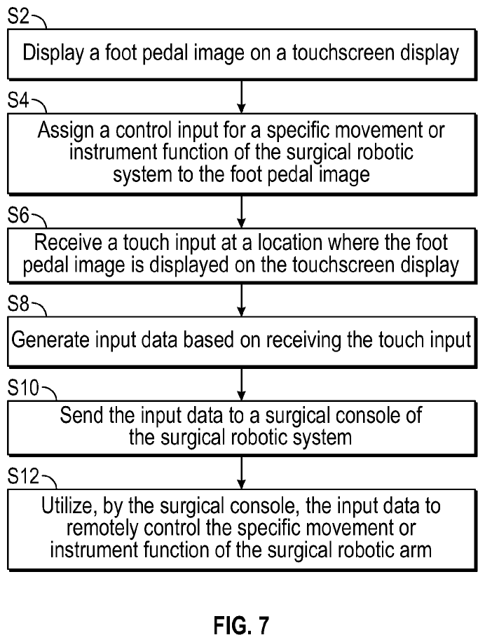

FIG. 7 illustrates a flow diagram of a method for controlling a movement or instrument function of the robotic arm 40 of the surgical robotic system 10. The method may include one or more operations, actions, or functions as illustrated by one or more of blocks S2, S4, S6, S8, S10, and/or S12. Although illustrated as discrete blocks, various blocks may be divided into additional blocks, combined into fewer blocks, or eliminated, depending on the desired implementation.

Processing may begin at block S2, “Display a foot pedal image on a touchscreen display.” At block S2, a graphic user interface (e.g., graphic user interface 36a) may display a foot pedal image (e.g., selectable foot input 36i) on a touchscreen display (e.g., touch screen display 36d) of the graphic user interface. The foot pedal image may include various shapes, sizes, colors, drawings, pictures, locations, orientation, and arrangements within the touch screen display. The foot pedal image may include left and right foot images, toe and heel images, drawings or pictures of a piece of equipment to be controlled, a power symbol, geometric shapes including boxes, circles, ellipses, triangles, and various shape/color/size combinations. The foot pedal image may further include text or indicia which may provide context or description to an activity controlled by the foot pedal image.

Processing may continue from block S2 to block S4, “Assign a control input for a specific movement or instrument function of the surgical robotic system to the foot pedal image.” At block S4, a processor (e.g., processor 36p) of the graphic user interface may assign a control input to the foot pedal image. The assigned control input may be for a specific movement or instrument function of the surgical robotic system.

Processing may continue from block S4 to block S6, “Receive a touch input at a location where the foot pedal image is displayed on the touchscreen display.” At block S6, the touchscreen device of the graphic user interface may receive a touch input at a location where the foot pedal image is displayed on the touchscreen device. The touch input may be a tap, a swipe, a touch and hold, and/or a slide.

Processing may continue from block S6 to block S8, “Generate input data based on receiving the touch input.” At block S8, a processor of the graphic user interface may generate input data based on receiving the touch input.

Processing may continue from block S8 to block S10, “Send the input data to a surgical console of the surgical robotic system.” At block S10, the processor of the graphic user interface may send the input data to a surgical console of the surgical robotic system.

Processing may continue from block S10 to block S12, “Utilize, by the surgical console, the input data to remotely control the specific movement or instrument function of the surgical robotic arm.” At block S12, the surgical console may utilize the input data to remotely control the specific movement or instrument function of the surgical robotic arm. Instrument function remotely controlled by input data from the graphic user interface may include bipolar coagulation, tissue cutting, stapling, monopolar power level, ultrasonic power level, etc.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

1-15. (canceled)

16. A foot pedal system for a surgical robotic system, the foot pedal system comprising:
a graphic user interface, wherein the graphic user interface is configured to:
display a selectable foot input on a touchscreen display;
assign a control input for a specific movement or instrument function of the surgical robotic system to the selectable foot input;
receive a touch input at a location where the selectable foot input is displayed on the touchscreen display;
generate input data based on receiving the touch input; and
send the input data to a surgical console of the surgical robotic system.

17. The foot pedal system of claim 16, wherein the graphic user interface includes a processor, a memory, a transmitter and the touchscreen display.

18. The foot pedal system of claim 16, wherein the selectable foot input includes shapes, colors, drawings, or pictures within the touchscreen display.

19. The foot pedal system of claim 18, wherein the selectable foot input includes at least one of a left foot image, a right foot image, a toe and heel image, or a drawing or picture of a piece of equipment to be controlled.

20. The foot pedal system of claim 18, wherein the selectable foot input includes text.

21. The foot pedal system of claim 16, wherein the graphic user interface provides an indication that a touch input has been registered.

22. The foot pedal system of claim 21, wherein the indication that a touch input has been registered is haptic feedback, audio feedback, visual feedback, or combinations thereof.

23. The foot pedal system of claim 16, wherein the touch input is at least one of a tap, a swipe, touch and hold, or a slide.

24. The foot pedal system of claim 16, wherein the input data is utilized by the surgical console of the surgical robotic system to remotely control the specific movement or instrument function of a surgical robotic arm.

25. The foot pedal system of claim 24, wherein the instrument function includes at least one of bipolar coagulation, tissue cutting, stapling, monopolar power level, or ultrasonic power level.

26. A system for controlling a movement or instrument function of a robotic arm of a surgical robotic system, the system comprising:
a touchscreen display;
a surgical console; and
the robotic arm;
wherein the touchscreen display is configured to output a graphic user interface including a selectable foot input, the graphic user interface configured to:
assign a control input for a specific movement or instrument function of the surgical robotic system to the selectable foot input;
receive a touch input at a location where the selectable foot input is displayed on the touchscreen display;
generate input data based on receiving the touch input; and
send the input data to a surgical console of the surgical robotic system,
wherein the surgical console utilizes the input data to remotely control the specific movement or instrument function of the robotic arm.

27. The system of claim 26, wherein the graphic user interface includes a processor, a memory, a transmitter and the touchscreen display.

28. The system of claim 26, wherein the selectable foot input includes a plurality of shapes, sizes, colors, drawings, pictures, locations, orientations, and arrangements within the touchscreen display.

29. The system of claim 26, wherein the graphic user interface includes a mode for movement of selectable foot inputs to move a selectable foot input within the touchscreen display with a motion of a user's foot on the touchscreen display.

30. The system of claim 29, wherein the mode for movement of selectable foot inputs allows a user to move a selectable foot input within the touchscreen display based on a motion of a user's hands at a console display such as a graphic user interface of a surgeon's console or a graphic user interface of a control tower.

31. The system of claim 26, wherein the touchscreen display includes multiple touchscreen displays at different heights.

32. The system of claim 31, wherein the graphic user interface includes configurable software to move a selectable foot input from one touchscreen display to another touchscreen display.

33. The system of claim 26, wherein the touch input is at least one of a tap, a swipe, touch and hold, or a slide.

34. The system of claim 26, wherein the instrument function includes control of an endoscope or endoscopic camera.

35. A method for controlling a movement or instrument function of a robotic arm of a surgical robotic system, the method comprising:
displaying a graphic user interface including a foot pedal image on a touchscreen display;
assigning a control input for a specific movement or instrument function of the surgical robotic system to the foot pedal image;
receiving, at the graphic user interface, a touch input at a location where the foot pedal image is displayed on the touchscreen display;
generating input data based on receiving the touch input;
sending the input data to a surgical console of the surgical robotic system; and
utilizing, by the surgical console, the input data to remotely control the specific movement or instrument function of the robotic arm.
